(19) United States
(12) Patent Application Publication
(10) Pub. No.: US 2009/0062828 A1
(43) Pub. Date: Mar. 5, 2009

(54) MAGNETIC FIELD-BASED COLLOIDAL ATHERECTOMY

(75) Inventor: David W.M. Marr, Golden, CO (US)

Correspondence Address: SHERIDAN ROSS PC, 1560 BROADWAY, SUITE 1200, DENVER, CO, 80202, US

(73) Assignee: COLORADO SCHOOL OF MINES, Golden, CO (US)

(21) Appl. No.: 12/203,744

(22) Filed: Sep. 3, 2008

Related U.S. Application Data
(60) Provisional application No. 60/969,839, filed on Sep. 4, 2007.

(51) Int. Cl. A61B 17/22; A61M 1/00; A61N 2/00

(52) U.S. Cl. 606/159; 604/891.1; 600/12

(57) ABSTRACT

Methods, devices, and systems for performing a non-invasive form of angioplasty are provided. The device may include one or many magnetically controlled colloidal particles that can be used to scrub the interior walls of arteries or the like. The colloidal particles may be organized in any number of configurations and may also be moved in any number of ways in an effort to maximize the amount of plaque removed from the artery.

### CROSS-REFERENCE TO RELATED APPLICATIONS

This Application claims the benefit of U.S. Provisional Application No. 60/969,839, filed Sep. 4, 2007, the entire disclosure of which is hereby incorporated herein by reference.

This application is also related to U.S. patent application Ser. No. 10/711,767, filed on Oct. 4, 2004, which is a divisional of application Ser. No. 10/138,799, filed on May 3, 2002, now U.S. Pat. No. 6,802,489, which is a non-provisional of Application Nos. 60/288,346 and 60/289,504 filed on May 3, 2001 and May 8, 2001, respectively, all of which are incorporated herein by reference in their entirety.

### FIELD OF THE INVENTION

The present invention is directed toward methods and devices for manipulating one or many colloidal particles.

### BACKGROUND

Diseases caused by hardening of the artery due to formation of plaque, atherosclerosis, are the leading cause of illness and death in the United States. FIG. 1 depicts an exemplary artery that has been hardened due to the formation of plaque on its wall. To avoid bypass surgery, coronary angioplasty was developed in the late 1970's and is currently performed on over 1 million people per yr in the US. Though considered a relatively safe procedure, there are significant costs, risks and discomfort associated with invasive techniques which require the insertion of catheter.

### SUMMARY

It is our belief that current balloon-type angioplasty approaches could be performed instead through an injectable colloidal solution controlled and driven through the application of an external field. In this, individual particles will self-assemble into small devices capable of “roto-rooting” plaque from arterial walls within flowing vessels. Unlike balloon-type angioplasty, this approach would gently remove plaque, avoiding possible restenosis or the need for subsequent blood-thinning medication when drug-eluting stents are employed. In accordance with at least some embodiments of the present invention, individual particles may be injected in solution and, upon application of the appropriate field, self-assembled into small devices capable of removing plaque from arterial walls within flowing vessels.

As both the microdevice assembly and driving forces are provided by the external field, once the procedure is finished, devices “self-disassemble” into small building blocks readily removable by the body via phagocytosis. Though reminiscent of science fiction and nanobots capable of circulating throughout the body and cleansing us from unwanted pathogen and disease, we have already demonstrated within the laboratory the assembly, disassembly, and function of the miniature devices we propose.

Specifically paramagnetic colloidal particles are used which demonstrate a variety of advantageous properties: they are readily available in a variety of relevant sizes, they can be controlled with an external magnetic field, and their surfaces can be chemically modified making them physiologically well tolerated (having no measurable toxicity index in the studies that have been conducted). In fact, due to these factors, such systems are being used as contrast agents in magnetic resonance imaging and as the basis of targeted drug delivery and/or hyperthermia in cancer treatment applications. Here however, we will restrict our investigations to a size range that limits transport of the colloidal particles within the circulatory system, both so that they can be readily directed to the desired arterial location for treatment and so that they remain available for removal by the body's own defense mechanisms once the procedure is finished. In this proposal and based on what we have observed in preliminary investigations, we will study, in vitro, the efficiency of plaque removal as well as the ability to direct self assembled devices to specific locations within model vascular networks.

A majority of myocardial infarction is caused by a rupture of plaque which often leads to sudden death of victims who are apparently healthy and without prior symptoms. Plaque is made up of fat, cholesterol, calcium, and other substances found in the blood, components that are not typically homogeneously distributed. Plaques typically having a lipid-rich core and a thin fibrous cap, a complicated morphology that has made detailed understanding and modeling of the rupture process difficult. Clinically, plaque buildup is commonly treated through balloon catheterization, a procedure used to physically expand the artery, with or without mechanical stents, by pressing the plaque buildup against the arterial wall. A less invasive procedure, one which could be easily performed and focused on the cleaning of arterial walls over larger network regions, could be more effective in the longer term and lead to lower health care costs. Embodiments of the present invention provide the use of micron-scale beads for the removal of plaque and the scrubbing of the interior walls of vascular systems. Based on preliminary investigations where it has demonstrated that paramagnetic particles can be readily assembled into microdevices of varying function, it is believed that bead-based systems can provide an easily-employed and relatively inexpensive approach that is minimally invasive with little patient discomfort. The advantages in using these systems are based on two primary physical traits. The first is their small size—the colloidal systems we propose to employ here are between 2 and 8 μm in diameter and therefore of size comparable to the various blood components associated with natural vascular environments. As individual particles, they therefore can be readily injected into and pass within the vascular system. The second is that these particles can be assembled and manipulated with applied external fields, a fact that we have taken great advantage of in previous engineering-focused investigations to create functional microdevices such as pumps and micromixers.

These simple and basic physical properties that make them attractive however are complemented by a significant number of other advantages that have led to their current use in a variety of in vivo bio-based applications. For example, significantly smaller, nanoscale, paramagnetic systems are currently available as contrast enhancers in magnetic imaging applications. In addition, there is significant study now in the use of these as agents for hyperthermia approaches to cancer treatment. Here, both the ability to localize and heat them using non-invasive magnetic fields makes their use promising. Both of these current applications have demonstrated how well these systems are tolerated by the body; in fact, no measurable LD₅₀ has been found in investigations aimed at determining the toxicity of dextran-coated magnetite.

It is thus one aspect of the present invention to utilize a syringe to inject discrete and dispersed beads in the vascular system. Stable colloidal particles in the micron-size range will be used here—large enough to be effective on vascular length scales and to remain in circulation (>100 nm) yet small enough to be easily injected.

It is another aspect of the present invention to utilize an applied magnetic field to concentrate these discrete particles in targeted regions of the vascular system. Because these particles are paramagnetic, they become magnetic themselves when in the presence of a magnetic field and will translate via magnetophoresis in designed field gradients. Depending on applied external field, the approach can be tuned from relatively un-localized prophylactic procedures to highly-targeting angioplasty mimics. Though a focus can be based here on field-based localization, it is possible that surface functionalization of the colloidal building blocks could be used to target plaque chemically via weak bonding before field-based physical rooting is “turned on”.

It is still another aspect of the present invention to utilize the magnetic field to assemble these colloidal building blocks into micron scale devices that will rotate at designed rates and slowly remove material from plaque buildup. Note that the magnetic fields required for device function are small (0.005 T) relative to those employed during typical magnetic resonance imaging (1-3 T). Functioning like micro-rooters, the plaque removal rate will be controlled by the device size, concentration, and applied field strength.

It is yet another aspect of the present invention to simultaneously monitor the angioplasty process with magnetic imaging techniques. Given the enhanced magnetic contrast associated with these systems, it will be possible for monitoring the procedure, in vivo, via magnetic resonance imaging or angiography. Currently there is a need for tools and techniques that can be employed in high-magnetic field environments. Note here that typical magnetic imaging is performed at radio frequencies, values vastly higher than those employed for colloidal-device function and allowing for simultaneous operation.

It is still another aspect of the present invention that when the external field is removed, the devices are adapted to immediately disassemble into individual bead building blocks which can be removed from the vascular system. By avoiding the use of nano-sized paramagnetic systems, the micro-rooters are not deeply embedded within cells for example and instead remain within the vascular system. Therefore, and because particle surface chemistry and coating (with dextran for example) can be readily modified, controlled phagocytosis via the reticulo-endothelial system will lead to bead removal once the procedure is finished.

Clearly, colloidal systems have a number of advantages making it feasible to take simple, injectable particle building blocks and assemble them into functional microdevices. It has already been demonstrated by using applied field-based techniques to create colloidal-based microdevices, making pumps, valves, and even cell/particle separation devices within microfluidic systems. For use within the body however, previous techniques would require invasive approaches for light delivery and, to avoid this, here the use of magnetic fields is proposed for device assembly and actuation. In addition to being benign to the body, such fields lend themselves well to massive parallelization; a system capable of operating a single device will be equally capable of running as many devices as can be placed within the available field. In fact, this approach will allow assembly, vast parallelization, and simultaneous operation of millions of microdevices for a clean sweep of the vascular system if desired. With even a 1% colloidal solution having tens of millions of beads/ml the approach provides the necessary building blocks for device fabrication.

Finally, the beads we will employ here, and those used in current applications, generally comprise polystyrene, providing an easily synthesized and surface-modifiable matrix material. The approach we describe here however could be combined with bead-based techniques for targeted-drug delivery by using degradable matrices or other materials in which desired chemical treatments could be embedded. Very much analogous to the newly-available drug-eluding stents, magnetic field-based colloidal targeting of arterial plaque could combine mechanical with pharmacological treatment with the synergy and associated greatly increased efficacy.

In accordance with at least some embodiments of the present invention, an angioplasty device is provided that comprises a plurality of paramagnetic colloidal particles controllable by an external magnetic field and operable to be formed into a microdevice, wherein the microdevice is operable to be passed through a vascular system to remove materials embedded in the vascular system.

In accordance with at least some further embodiments of the present invention, a method of operating an angioplasty device is provided that comprises:
providing a plurality of paramagnetic colloidal particles;
organizing the plurality of paramagnetic colloidal particles into a microdevice; and
applying a magnetic field to the microdevice such that the microdevice is propagated through a blood vessel.
   These and other advantages will be apparent from the disclosure of the invention(s) contained herein. The above-described embodiments and configurations are neither complete nor exhaustive. As will be appreciated, other embodiments of the invention are possible using, alone or in combination, one or more of the features set forth above or described in detail below.

### DESCRIPTION OF THE DRAWINGS

FIG. 1 depicts a diagram of plaque and associated atherosclerosis in an artery;

FIG. 2 depicts a pump design with 30 degree rotation steps to illustrate lobe movement in accordance with at least some embodiments of the present invention;

FIG. 3 depicts colloidal silica used as a peristaltic pump in accordance with at least some embodiments of the present invention;

FIG. 4 depicts an actuated three-way colloidal valve in accordance with at least some embodiments of the present invention;

FIG. 5 depicts the self-assembly of seven magnetic particles in a channel structure into a compact micropump in the presence of an external rotating magnetic field in accordance with at least some embodiments of the present invention;

FIGS. 6A-6D depict pumping using a translating colloidal assembly in accordance with at least some embodiments of the present invention;

FIGS. 7A-7C depict mixing within a microchannel network using rotating paramagnetic colloidal assemblies in accordance with at least some embodiments of the present invention;

FIG. 8 depicts a microfluidic network in accordance with at least some embodiments of the present invention;

FIGS. 9A-9B depict the various sizes of assemblies that can be utilized in accordance with at least some embodiments of the present invention;

FIG. 10 depicts a cross-sectional view of FIG. 8 in accordance with at least some embodiments of the present invention.

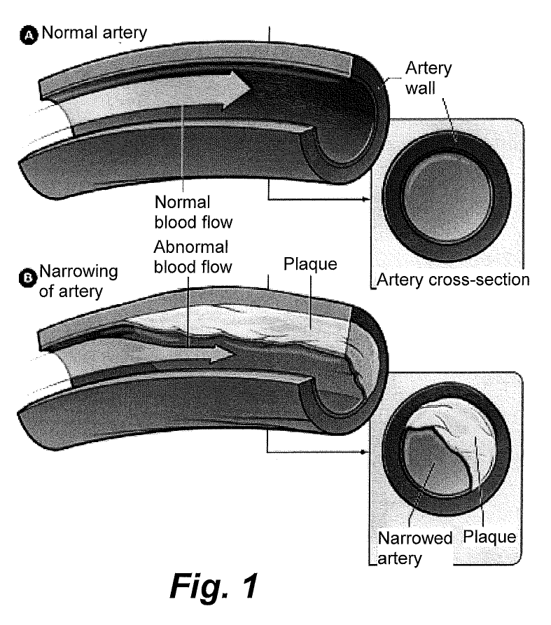

### DETAILED DESCRIPTION

### Colloidal Devices

Functional devices have already been developed out of colloidal systems at length scales significantly smaller than previously achieved by other techniques. Embodiments of the present invention have successfully created gear pumps, peristaltic pumps, and two-way valves, all at sizes that approximate those of a human red blood cell. Details of such devices are further discussed in U.S. Patent Publication No. 2005/0175478, the entire contents of which are hereby incorporated herein by this reference. The primary advantage of doing microfluidics at such small scales is that vastly smaller quantities are required than is needed for current technologies; however, additional advantages rely on the unique, viscosity-dominated, nature of the fluid dynamics at these sizes. In these, we have manipulated colloids by optical trapping, a non-contact, non-invasive technique that eliminates the need to physically interface to the macroscopic world, thus circumventing a traditional obstacle to microfluidic device miniaturization. The optical trapping principle is based upon a focused laser beam encountering a colloid of refractive index different than its surrounding solvent, causing the particle to reflect and refract the focused beam. Such photon redirection must be balanced by a change in colloid momentum, the net result of which is the trapping of small micron-sized objects in the focal point of a converging laser beam. In order to manipulate complex asymmetric objects or multiple objects at once, as is required for the actuation of a microfluidic pump, a great number of optical traps are simultaneously required. To accomplish this, we have employed a scanning approach in which a piezoelectric mirror is translated to rapidly reflect a laser beam in a desired pattern. If the piezoelectric mirror is scanned over the desired pattern at a frequency greater than that associated with Brownian time scales, a time-averaged trapping pattern is created. The details of this approach, called scanning laser optical trapping (SLOT), can be found elsewhere.

Under microfluidic conditions where viscous effects dominate, the fluid dynamics are unique. The Reynolds number, defining the ratio of inertial to viscous forces, is very small reducing the equations of motion to a simple time reversible differential form known as the Stokes equation. These microfluidic flows are completely dominated by viscous effects and are therefore laminar in nature, time reversible and turbulent free. The physical nature of these microfluidic flows determines the approaches one can use in designing both microscale pumps as well as microscale mixers which relies on diffusion.

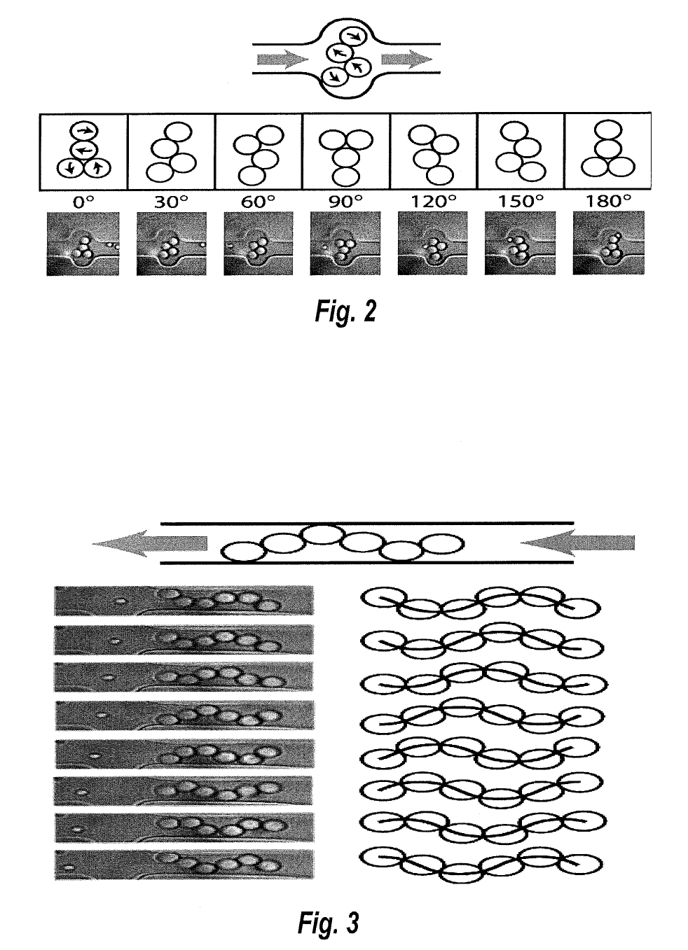

One design is a two-lobe gear pump in which small, trapped pockets of fluid are directed through a specially-designed cavity fabricated in a microchannel by rotating two colloidal dumbbells or “lobes” in opposite directions. Over repeated and rapid rotations, the accumulated effect of displacing these fluid pockets is sufficient to induce a net flow. This motion is illustrated in FIG. 2, where clockwise rotation of the top lobe combined with counterclockwise rotation of the bottom lobe induces flow from left to right. In the experiments also shown in FIG. 2, each of the lobes consisted of two, independent 3 μm silica spheres. To create these structures, the colloids were first maneuvered using the optical trap to a 3 μm deep section of channel designed with a region of wider gap to accommodate lobe rotation. The rotation of the lobes may occur at a rate of about 2 Hz. Once the particles were properly positioned, the laser was scanned in a manner such that a time-averaged pattern of four independent optical traps was created, one for each microsphere comprising the two-lobe pump. By rotating the two traps in the upper part of the channel and the two traps in the lower part of the channel in opposite directions and offset by 90°, the overall pump and the corresponding fluid movement was achieved. Flow direction was easily and quickly reversed by changing the rotation direction of both top and bottom lobes.

The gear pump design illustrates the success of positive displacement pumping through the use of colloidal microspheres; however, its design may prove particularly harsh to certain solutions. Though individual cells can be pumped using the gear pump, concentrated cellular suspensions may be damaged by the aggressive motion of the meshing “gears” of the pump. A second approach has been developed that incorporates a peristaltic design also based upon the concept of positive fluid displacement, effectively a pseudo two-dimensional analog of a three-dimensional, macroscopic screw pump. If instead of rotating the particles as in the gear pump, they are translated back and forth across the channel in a cooperative manner, fluid propagation can be achieved. One main advantage of this peristaltic design lies in the simplified, reciprocal motion of the microspheres, which may allow actuation by other methods such as electrophoresis.

The colloidal movement required to direct flow via the peristalsis approach is illustrated in FIG. 3. The optical trap moves the colloids in a propagating sine wave within which a plug of fluid is encased. Direction of the flow can be reversed by changing the direction of colloidal wave movement. Once again, these experiments were performed with independent, 3 μm silica spheres; however, more colloids were used in the experiments of FIG. 3 to represent a complete wavelength. Fabrication of these pumps required first maneuvering the colloids into the channel section. Once in place, the optical trap was scanned such that multiple independent traps were created, one for each colloid compromising the peristaltic pump.

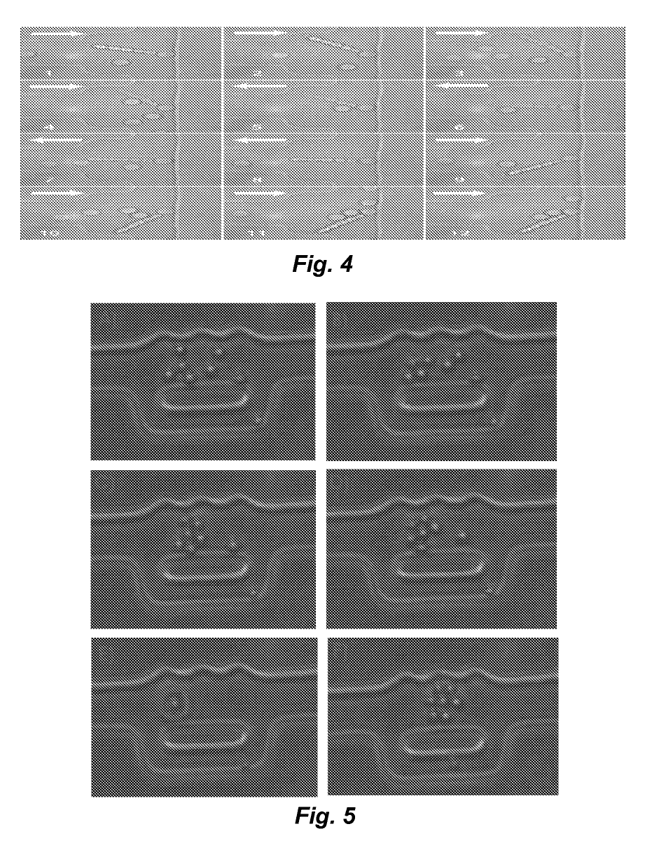

In addition to pumps, simple valves can be created using a similar technique. These are shown FIG. 4, where the valves consist of a 3 μm silica sphere photopolymerized to several 0.64 μm silica spheres forming a linear structure. For passive application, the device was maneuvered into a straight channel and the 3 μm sphere held next to the wall allowing the arm to rotate freely in the microchannel. As the flow direction is changed, the valve selectively restricted the flow of large particles in one direction while allowing passage of all particles in the other. To actively direct particulates to one of two exit channels, the passive valve was maneuvered into a confining T geometry. As the valve structure was rotated about its swivel point using the optical trap, the top or bottom channel was sealed, directing flow of particulates toward the open channel in FIG. 4.

These results clearly demonstrate that microscale devices composed of simple colloidal building blocks can perform complex functions such as pumping and valving.

### Paramagnetic Colloidal Devices—Pumps

Over the past decade there has been a tremendous growth in the use of microfluidic systems for a variety of proposed applications. A good review of the current state of the art, the most pressing needs, as well as some of the more promising applications can be found in a recent issue of Nature. Because magnetic fields are relatively simple to generate and provide the possibility of transferring energy across length scales without direct contact, such fields may solve some of the issues preventing wide-scale adoption of microfluidic technologies. As such, previous microfluidic studies have employed magnetic fields for separations of cells, separations using superparamagnetic particles such as those we employ here as well as pumps. Though our approach differs greatly from other studies in that we are creating very small-scale devices of distinct local function for operation within the body, our goal is similar—to develop approaches to the operation of microfluidic devices that are simple yet both capable and practical.

As discussed previously we intend to develop complementary magnetic field manipulation techniques to aid the assembly and operation of our fluidic-based colloidal microdevices. For our preliminary investigations, Dynabeads® (www.dynalbiotech.com) of diameter either 2.7 μm or 4.5 μm were used. Developed for bioassaying applications, these readily-available particles are superparamagnetic due to the presence of Fe₂O₃ and therefore exhibit magnetic properties only in the presence of a magnetic field. They are available at low polydispersities making them a convenient model system for our investigations. Our microfluidic systems are planar in nature and have been fabricated such that channel height is typically little more than the particle diameter. This confinement plane provides the reference point for the application of our external magnetic fields. Here coils are placed in this same plane and external to the entire microfluidic device. Upon application of current through these coils a magnetic field is created that induces an effective attraction between Dynabeads®. As the polarization of the magnetic field is rapidly rotated using the three distinct coils, a torque is induced that can be used to rotate these colloidal assemblies. In this setup, an optical trap has been included for ease of particle manipulation.

The frames depicted in FIG. 5 demonstrate the assembly of seven 4.5 μm particles into a compact rotating cluster in the presence of a rotating magnetic field. It has been shown previously that slow field rotation frequencies lead to the formation of chain-like structures which rotate around their center of mass. We observe however that when the clusters are located inside channels, compact structures independent of the rotation frequency are always favored. Note that cluster formation in these systems can be either reversible or irreversible depending on specific colloid surface chemistry and strength of the applied magnetic field.

Application of a rotating magnetic field to a compact particle cluster leads to a cluster rotation rate dependent on a balance between viscous drag and the magnetic forces. FIG. 5 depicts frames of a sequence showing a rotating cluster composed of seven particles in a microchannel structure filled with water. The external field rotates at a frequency of 100 Hz in the plane of the particles in a counter-clockwise direction. It induces a torque on the cluster due to the interaction with the magnetic dipoles and leads to a cluster rotation. In the case of the 7 particle cluster shown here this leads to a maximum cluster rotation frequency of approximately 20-30 Hz. Due to the length scales of our microfluidic channels and pumps, flow is laminar and a rotating particle cluster can only induce a net flow if the channel symmetry is broken. We therefore fabricated the channel walls with depressions on one side. When applying a rotating field, the cluster aligns itself close to the curved side of the channel. This becomes apparent when considering the flow created by the pump as shown by observing the motion of tracers. Here, the pump is situated in a structured channel, with a maximal width of approximately 16 μm and height 6 μm. Flow was visualized by the motion of non-magnetic polystyrene tracer particles and it can be seen that pumping increases with the strength of the rotating field. This is measured by taking the time the tracer needs to pass the bypass for different field strengths. In fact, because the pumps can rotate very rapidly, we have been unable to quantify the exact rotation speed in the preliminary setup.

In these studies we created pumps of two, three and seven particles in similar geometries of varying channel width as well as pumps connected in series. It has been found that the pump efficiency increases with increasing pump diameter. More particles have a bigger collective magnetic moment, an effect that leads to faster rotation for a given applied magnetic field. These larger clusters also have more surface leading to a stronger hydrodynamic interaction with the surrounding fluid. In addition, pumps connected in series lead to larger flows than single pumps. In this, the external application of the magnetic field, leads to reversible aggregation of smaller numbers of paramagnetic colloidal spheres. Seven such particles in a confined, two-dimensional geometry such as we have here typically leads to a flower-like cluster. In this image, two such clusters have been formed and, as the magnetic field is rotated, these cluster rotate as well. Though certainly better seen in movie clips not available here, the cluster rotation leads to fluid flow; this is verified by changing the rotation direction of the clusters where the tracer now moves in the opposite direction. Using larger 4.5 μm Dynabeads we have achieved rapid rotation rates of at least 5 Hz, significantly increasing fluid flow velocities.

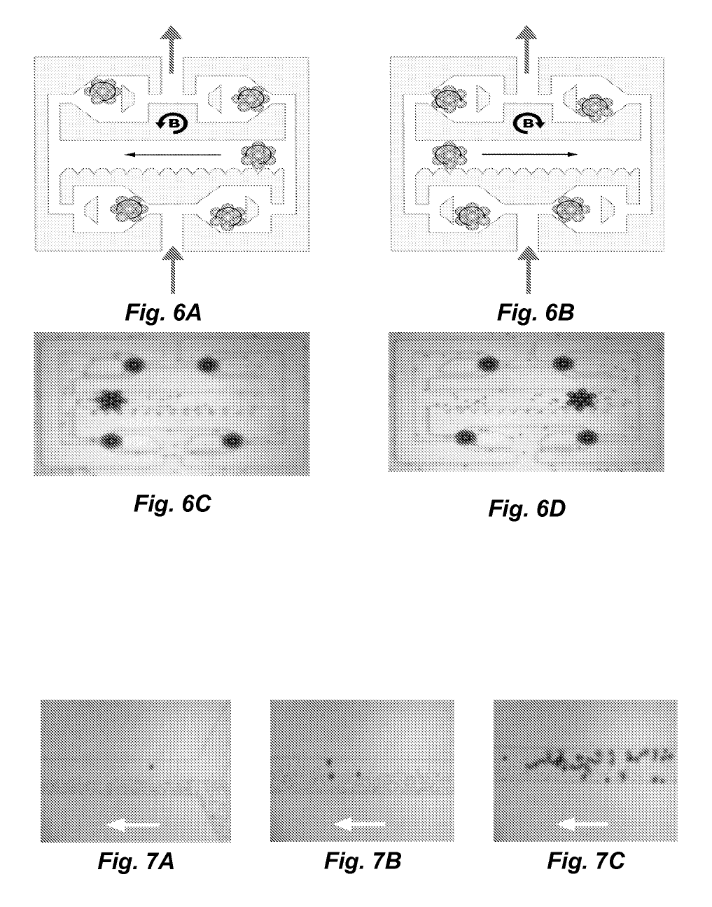

In our studies, the channels were designed to capture the pump in the asymmetric part and prevent translation along the wall because of the strong interaction between walls and cluster. This interaction can induce a small circular movement of the pump center of mass, which has no observable influence on the pump efficiency. In addition however, the microchannel design plays an important role in device function. As these devices are powered using an external source, their rotation is driven in the same direction, a feature that, at first glance, may limit function. However, pumping direction depends both on the cluster rotation direction and the channel geometry. As illustrated in FIGS. 6A-6D for pumps rotating in identical directions, net flow is determined by the location of the channel asymmetry. Although pump assembly and powering are driven by the external field, pumping direction is dictated by the physical geometry in which the pump is fabricated.

The approach discussed here allows the simultaneous creation of large numbers of micropumps inside microfluidic devices. We have demonstrated this with six three-particle pumps composed of 2.7 μm particles which rotate in the same direction at approximately the same speed. Though certainly more dense configurations are possible, this image corresponds to a pump density of approximately 30,000 pumps/cm². Note that the energy required to drive all of these individual devices simultaneously is provided by a single external source. Despite the large number of available pumps and the ability to direct pumping with static channel designs, more dynamic control is of interest for some applications. In our approach, a global field is used to power all of the individual devices simultaneously; however, local modifications to the field or application of a separate, supplementary field, can alter local function.

In accordance with at least some embodiments of the present invention, both the assembly and operation of paramagnetic-colloid based microdevices can be controlled using magnetic fields completely external to the system.

### Paramagnetic Colloidal Devices—Mixers and the “Micro-Rooter”

It is well known and a significant area in microfluidics research that mixing in microscale geometries is difficult due to the laminar nature of the fluid flows, the associated lack of turbulence, and the resulting reliance on diffusion (see for example the review). Though approaches specific to flexible microfluidic networks have been developed, FIGS. 7A-7C show mixing within microchannel networks using rotating paramagnetic colloidal assemblies. These preliminary investigations show co-flowing non-mixing parallel flows (FIG. 7A) and the subsequent mixing upon addition of active colloidal devices (FIGS. 7B and 7C). Note here the very high level of parallelization.

Embodiments of the present invention have been employed to determine the feasibility of employing colloidal systems for plaque removal in vascular-like microscale systems. For these studies, microfluidic model networks based on PDMS will not only allow easy imaging of results, they allow creation of model networks.

As envisioned, colloidal solutions can be injected at low concentration where they will function in a highly-parallel fashion or directed to specific locations for plaque removal via applied external field where they will be assembled and their function switched on.

These microfluidic systems are assembled using a methodology coined “rapid prototyping”. In this, and using standard photolithography techniques, a pattern is produced on silicon or silicon dioxide substrates in thick SU-8 photoresist. Following the photolithography step, the pattern is then used directly as a “master” to produce positive relief replicas in PDMS, an optically transparent elastomer, a process that has come to be known as “soft lithography”. Specifically, templates of microchannels (μChs) and microfluidic networks (μFNs) are created lithographically with ultraviolet (UV) light by transposing the pattern of a shadow mask to a UV sensitive negative photoresist. The patterns are subsequently developed in an appropriate solution, leaving only the negative relief of the desired pattern, which may be used directly as a PDMS master or etched to produce a permanent master. If used directly to create PDMS replicas, photoresist films may be prepared with thickness from 25 nm to 250 μm, thus providing a wide range of accessible sizes and aspect ratios. Except for situations in which extremely thin films are required, SU-8 series negative photoresist (MicroChem Corp., Newton, Mass.) is employed, which is capable of producing rugged patterns with high aspect ratios that can be directly cast into PDMS replicas and reused many times.

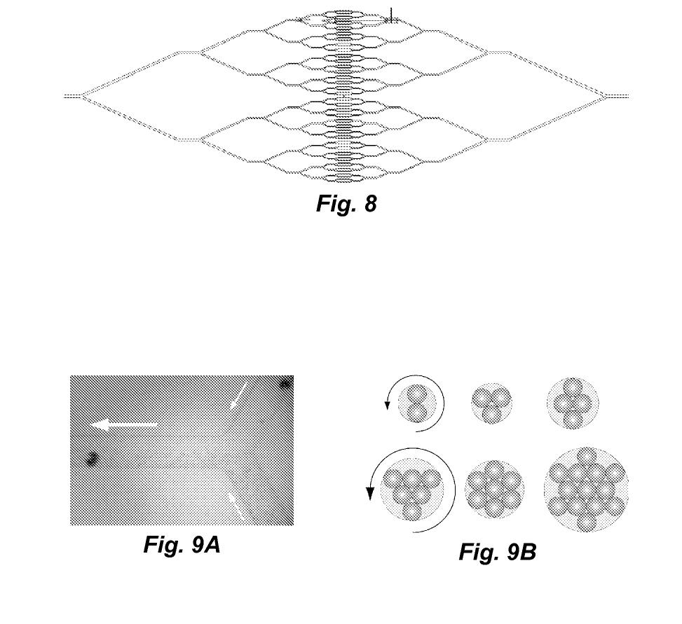

The PDMS replicas are created using a commercially available two-component kit (Sylgard 184 Kit, Dow Corning). A mixture of elastomer and curing agent are poured over the silicon master and cured under vacuum to degas the elastomer solution. PDMS makes an ideal candidate for μFN production because it can be cured quite rapidly, patterns are faithfully reproduced, even on the nanoscale and the process can be conducted in a non-clean room environment. Once cured, PDMS replicas are peeled from the master, leaving a clean, reusable template. The replica is finally placed in conformal contact with either a glass slide or PDMS flat forming a tight, reversible seal and enclosing channels capable of conveying fluids. PDMS is natively hydrophobic, but can be easily modified to create a hydrophilic surface through brief exposure to an oxygen plasma. Replica films as thin as 1 μm may also be created by spin coating PDMS onto a silicon master. Such films may be patterned and used as soft components such as micro gaskets, seals and spacers for multilevel functional devices. Thicker films (>40 μm) may be removed from the substrate and used as shadow masks for the deposition of metal features, such as electrodes, onto other replicas or a wet etching mask for the patterning of conducting tin oxides. FIG. 8 depicts an exemplary network that may be constructed in accordance with at least some embodiments of the present invention.

FIGS. 9A and 9B depict and Table 1 shows that clusters of 2, 4, and 6 particles may be particularly efficient at removing plaque from arterial walls due to their relatively higher circle area/particle ratio.

TABLE 1
Circle Area/Particle Size Ratio

| | | circle |
|---|---|---|
| particle # | circumradius | area/particle |
| 2 | 1.000σ | 2.00000 |
| 3 | 1.0774σ | 1.54772 |
| 4 | 1.3660σ | 1.86596 |
| 6 | 1.6547σ | 1.82535 |
| 7 | 1.5000σ | 1.28571 |
| 13 | 2.2321σ | 1.53295 |

One question addressed by embodiments of the present invention is whether plaque removal efficiency can be improved through magnetic-field modification. For example, with a rotating field, device can be assembled and rotated; however, these will tend to remain within their streamlines (in the middle of flow) and not translate to arterial walls. This is both due to the high Pe and lack of Brownian diffusion in the assembled aggregates as well as a tendency for particulate systems to remain in regions of lower shear (and not the highest shear wall region in these pressure driven flows). Though certainly device location will be impacted (and to some extent randomized) by its rotation, we intend to determine how the field can be modified to enhance transport in directions lateral to flow. One very simple approach is to apply small gradients in the field that slowly vary in a sinusoidal fashion, effectively and gently pushing devices from one wall to another. Because the field is, by design, always rotating, the gradient direction can readily varied over time, either randomly or in a smoothly changing fashion.

One approach may employ a field that rotates continuously in one direction leading to a very rapid spin of the colloidal devices. Plaque removal however may be more rapidly accomplished with devices that instead rotate back and forth (i.e. rotate one direction and then the other). Because of the ease with which the external field is modified such alternative motions may be investigated.

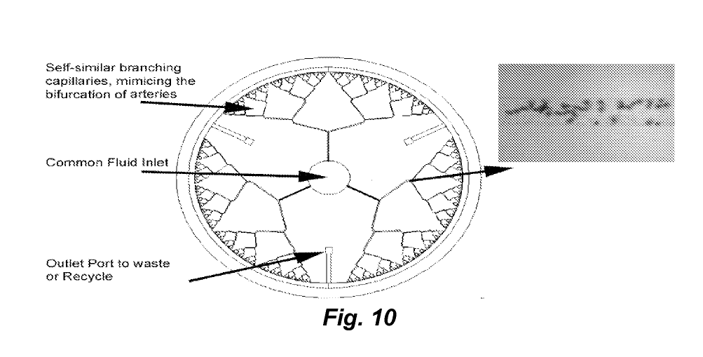

For preventative treatments weak localization of particle systems may be desired. Here fields will be generated to isolate beads within larger regions (modeling the leg or the coronary region for example). To drive net particle translation, weak, low-frequency (including zero frequency) magnetic field gradients will be applied through a combination of anisotropic current through the differing coils and through slight experimental modifications including the use of soft-iron cores. For these studies, microfluidic geometries, as illustrated in FIG. 10, which allow investigation of transport through networks will be employed. These geometries are easily modified by making a new mask and re-fabricating and can be investigated with or without applied flows.

Strong localization as an angioplasty mimic may also be produced. Here and using overall more dilute colloidal suspensions, stronger field gradients will be needed to create highly local regions capable of device assembly. More closely mimicking current angioplasty procedures, strong localization may be needed for situations where a specific target plaque has been identified for dispersal. Because of the reduced number of required colloidal particles required however, strong localization (but with translation) may be a preferred technique in some applications. In these studies, we will test the conditions necessary for both. Previous investigations with the goal of manipulating single, small Dynabeads in solution with an integrated microscope have demonstrated that very strong gradients (˜1 T/cm) can be externally created in these systems. Because our localization requirements are not nearly as stringent and we will be working with beads and devices of significantly greater saturated magnetic moments, this technique, known as “magnetic tweezing”, requires much higher fields than we will require but does demonstrate the capabilities inherent in the approach.

In accordance with at least some alternative embodiments of the present invention, if desired localization is not achieved in reasonable time scales using the moderate fields, local permanent magnets or magnetizable materials may be employed. Studies of tumor targeting with paramagnetic drug-delivering particles have used this approach in vivo to enhance particle delivery driven through external magnetic fields. To avoid losing many of the advantages inherent in our specific approach, materials may be employed externally but in close proximity to the microfluidic device.

The present invention, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An angioplasty device, comprising:
a plurality of paramagnetic colloidal particles controllable by an external magnetic field and operable to be formed into a microdevice, wherein the microdevice is operable to be passed through a vascular system to remove materials embedded in the vascular system.

2. The device of claim 1, wherein the colloidal particles are organized such that the microdevice comprises at least one of a gear pump, a two-lobe gear pump, a peristaltic pump, a valve, and a two-way valve.

3. The device of claim 2, wherein the microdevice comprises the two-lobe gear pump and wherein the two-lobe gear pump comprises at least a first lobe and second lobe that are rotated in opposite directions relative to one another.

4. The device of claim 2, wherein the microdevice comprises the peristaltic pump and wherein the plurality of paramagnetic colloidal particles within the peristaltic are controlled by an optical trap that moves the colloidal particles in a propagating sine wave.

5. The device of claim 1, wherein the colloidal particles are formed into the microdevice by optical trapping.

6. The device of claim 5, wherein optical trapping comprises translating a piezoelectric mirror over a predetermined pattern thus resulting in at least one optical trap being scanned across the plurality of paramagnetic colloidal particles.

7. The device of claim 1, wherein the microdevice is driven through the vascular system by an externally applied magnetic field.

8. The device of claim 1, wherein at least one of the plurality of paramagnetic colloidal particles comprises polystyrene.

9. The device of claim 1, wherein at least one of the plurality of paramagnetic colloidal particles comprises a drug-delivery mechanism.

10. The device of claim 1, wherein at least one of the plurality of paramagnetic colloidal particles comprises a chemical that is soluble in the vascular system.

11. The device of claim 1, wherein the vascular system comprises a vascular system of a human patient.

12. A method for controlling and steering paramagnetic colloidal particles, comprising:
providing a plurality of paramagnetic colloidal particles;
organizing the plurality of paramagnetic colloidal particles into a microdevice; and
applying a magnetic field to the microdevice such that the microdevice is propagated through a blood vessel.

13. The method of claim 12, wherein the colloidal particles are organized such that the microdevice comprises at least one of a gear pump, a two-lobe gear pump, a peristaltic pump, a valve, and a two-way valve.

14. The method of claim 13, wherein the microdevice comprises the two-lobe gear pump and wherein the two-lobe gear pump comprises at least a first lobe and second lobe that are rotated in opposite directions relative to one another.

15. The method of claim 13, wherein the microdevice comprises the peristaltic pump and wherein the plurality of paramagnetic colloidal particles within the peristaltic are controlled by an optical trap that moves the colloidal particles in a propagating sine wave.

16. The method of claim 12, wherein the colloidal particles are formed into the microdevice by optical trapping.

17. The method of claim 16, further comprising translating a piezoelectric mirror over a predetermined pattern thus resulting in at least one optical trap being scanned across the plurality of paramagnetic colloidal particles.

18. The method of claim 12, further comprising guiding the microdevice to through the vascular system via application of a magnetic field.

19. The method of claim 12, wherein at least one of the plurality of paramagnetic colloidal particles comprises polystyrene.

20. The method of claim 12, wherein at least one of the plurality of paramagnetic colloidal particles comprises a drug-delivery mechanism.

21. The method of claim 12, wherein at least one of the plurality of paramagnetic colloidal particles comprises a chemical that is soluble in the vascular system.

22. The method of claim 12, wherein the vascular system comprises a vascular system of a human patient.

23. The method of claim 12, further comprising applying an optical field to the plurality of paramagnetic colloidal particles to organize them into the microdevice.
